(19) United States
(12) Patent Application Publication
(10) Pub. No.: US 2014/0102459 A1
(43) Pub. Date: Apr. 17, 2014

(54) Expandable Inter Vivos Tube

(71) Applicants: Joseph A. Vilasi, Lakewood Ranch, FL (US); Joseph D'Ambrosio, Ridgefield, CT (US)

(72) Inventors: Joseph A. Vilasi, Lakewood Ranch, FL (US); Joseph D'Ambrosio, Ridgefield, CT (US)

(21) Appl. No.: 14/109,880

(22) Filed: Dec. 17, 2013

Related U.S. Application Data
(63) Continuation-in-part of application No. 13/662,552, filed on Oct. 29, 2012.
(60) Provisional application No. 61/705,959, filed on Sep. 26, 2012.
(60) Provisional application No. 61/911,589, filed on Dec. 4, 2013.

(51) Int. Cl. A61M 16/04

(52) U.S. Cl. CPC A61M 16/0434
USPC 128/207.15

(57) ABSTRACT

A flexible expandable inter vivos tube includes at least one arched segmented portion, a corresponding movable element and at least one expansion mechanism. The at least one arched segmented portion and corresponding movable element forming a flexible closed longitudinally expandable tube. The at least one arched segment includes an H-shaped connector forming cavities that allows variable slidable movement of a free end portion of the corresponding movable element. To move one or more of the longitudinal segments of the expandable tube, which are within the cul-de-sac cavities of the H-shaped connector, a sliding mechanism within the H-shaped connector moves from a first position to a second position to cause the expansion mechanism to expand such that the diameter of the flexible inter vivos tube is increased.

### CLAIM OF PRIORITY

This applicant claims, pursuant to 35 USC 119, priority to, and the benefit of the earlier filing date of Provisional US Patent entitled “Expandable Inter Vivos Tube, filed on Dec. 4, 2013 and afforded Ser. No. 61/911,589, and pursuant to 35 USC 120, the benefit of the earlier filing date, as a continuation-in-part, of that patent application entitled “Expandable Inter Vivos Tube,” filed in the US Patent and Trademark Office on Oct. 12, 2012 and afforded Ser. No. 13/662,552, which claimed priority to provisional patent application entitled “Expandable Inter Vivos tube,” filed in the US Patent and Trademark Office on Sep. 26, 2012, and afforded Ser. No. 61/705,959, the contents of all of which are incorporated by reference herein.

### BACKGROUND

1. Field of the Invention

The present invention relates to the field of medical devices and, more particularly, to an expandable tube for inter vivos used in medical procedures.

2. Background of the Invention

Inter vivos tubes, such as endotracheal tubes, are used to provide gases to the lungs during surgery. For example, an endotracheal tube is inserted into the trachea with its distal tip advanced halfway toward the tracheal bifurcation to provide gases, such as oxygen and anesthetics, to a patient during surgery. The exposed portion of the endotracheal tube is then firmly taped to the patient's face to prevent undesirable movement.

To align the position of conventional endotracheal tubes, an inflatable cuff balloon, at the distal end of the endotracheal tube, is inflated to correspond to the inner diameter of a portion of the trachea, thereby centering, or otherwise positioning, the endotracheal tube within the trachea. The cuff balloon, however, does not completely obstruct the entire trachea; only the portion where it is anchored is obstructed. When the cuff balloon is inflated, confirmation of the expanded balloon's contact within the trachea is achieved and delivery of anesthetic gases is performed.

Because of various sized endotracheal tubes, it is preferable to at least make the outer diameter of the endotracheal tube closely proximate to the size of the glottis, or opening between the vocal cords, for selective positioning of the endotracheal tube at a predetermined dilation. Therefore, various sized tubes are used, and the anesthesiologist or nurse anesthetist must choose from a variety of sized tubes to insert in the patient. If nasotracheal intubation or tracheostomy tubes are required in present practice even smaller interior diameters (ID) tubes are used.

Conventional endotracheal tubes vary in size and are numbered according to an internal diameter (ID). For example, for children, tubes are measured at about 3.5 to 7 mm (millimeters) internal diameter and from 7 to 11 mm for an adult. The internal diameter in women varies in general from 7.0 to 8.5 mm ID and in men from 8 to 10 mm ID. Typically, an endotracheal tube size selected for each patient is empirically selected by the anesthesiologist based on the patient's gender, age and size.

Ideally, the endotracheal tube should approximate as closely as possible the glottic size of the patient. Since there is no way to estimate the glottic size prior to the administration of anesthesia, in the existing prior art endotracheal tubes, a distal inflatable cuff is incorporated into the present day endotracheal tube which, when inflated, compresses the tracheal wall, thus creating a closed circuit between the endotracheal tube inflow from the anesthesia machine and outflow from the patient's lung to the exhalation valve. When nasotracheal intubation or tracheostomies is required, the internal diameter of the endotracheal tube is even less than the normal sizes, which are selected for orotracheal intubation, which results in even greater respiratory resistance.

Furthermore, as noted in “Clinical Anesthesia”, 1989 Edition, J. B. Lippincott Company, edited by Paul Barash, MD, Bruce Cullen, MD, and Robert Stoelting, MD, “[e]ndotracheal tube resistance varies inversely with the tube size. Each millimeter decrease in tube size is associated with an increase in resistance of 25 to 100%. The work of breathing parallels changes in resistance. A one (1) mm decrease in tube size increases the work of breathing from 34 to 154%, depending on the ventilatory pattern”.

Therefore, in existing prior art inter vivos tubes, the internal diameter is small, and the only large portion is the external cuff balloon. This makes it harder for a surgical patient to breathe through the small internal diameter of the existing endotracheal tubes, especially if the patient must breathe spontaneously without assistance.

In summary, the prior art uses a local, inflatable balloon at the distal portion of an endotracheal tube, which narrows the patient's air way at the vocal cord level and may damage the vocal chords of the patient, if not property installed. In addition, the inflated balloon may damage the vocal chords not properly deflated prior to extubation.

Applicant's prior U.S. Pat. No. 3,968,800 dated Jul. 13, 1976 and U.S. Pat. No. 4,827,925 dated May 9, 1989 describe an adjustable endotracheal tube which is complex to expand, and which does not have flexibility in being adapted to varying sized tracheas or vocal cord apertures of different patients. Applicant's other prior U.S. Pat. No. 4,722,335 dated Feb. 2, 1988 discloses an expandable endotracheal tube including two overlapping curved segments, which when joined together form a closed tube. Similarly, applicant's prior U.S. Pat. No. 5,647,358, dated Jul. 15, 1997, discloses an expandable inter vivos tube that provides for expansion of the tube along at least designated parts of the tube. However, the configuration may be conceptually possible but in practical terms, difficult to construct and maintain at present prices.

Hence, there is a need in the industry for a disposable expandable inter vivos tube that is easy to construct, easy to install, expand and remove during a procedure while reducing construction and costs of construction.

### SUMMARY OF THE INVENTION

It is therefore an object of the present invention to provide a flexible, expandable inter vivos tube that expands its internal diameter, at the glottic region of the trachea, to make breathing easier for a surgical patient.

Another object of the flexible, expandable inter vivos tube of the present invention is to vary a size of the internal diameter (ID) of an endotracheal tube in order to reach the glottic size of the patient without the intervention of a distal inflatable cuff. With the present invention, the distal cuff is unnecessary and a one-size endotracheal tube would fit most all adult patients. The present invention is especially useful in nasotracheal intubations where normally an even smaller internal diameter tube would be selected by the anesthesiologist. The expandable tube concept can also find usage with stainless steel spiral endotracheal tubes that may be used when lasers are used or for tracheostomies or laryngectomy tubes. It would also be find useful applications in laparoscopic surgery, for example, duration surgical interventions. Such expandable tubes would find applications as esophageal dilators, in cystoscopy procedures, as neurosurgical retractors, in thoracic surgery, aortic aneurysms, laparoscopic surgery, for example, diminishing the duration of surgery, and a myriad of other applications. An expandable laparoscope would further enable surgeons to engulf a tumor or cystic lesion rather than having to cut the tumor into pieces for extraction. Removal of tumors or cystic lesions in fairly large sizes results in shorter surgery times and reduced bleeding. Other expandable tube applications can include procedures involving cystoscopy, esophageal dilators, aortic aneurysms and other procedures that require expansion of an inserted tube.

It is also an object of the present invention to provide an endotracheal tube that maintains the same wall thickness throughout, without tapering.

It is yet another object of the present invention to provide an inter vivos tube having an internal diameter that remains substantially consistent from a proximal end to a distal end without the need for tapering, e.g., Colepediatric tubes, which maintain through a tapered design apply pressure on the vocal cords in order to establish a one-way flow. Basically two size expandable endotracheal tubes can be considered; pediatric size (expanding to 7 mm internal diameter) and adult size (which can expand up to 11 mm (or greater if necessary)). Any tapering of the inter vivos tube ultimately results in a restricted airway at the point of tapering.

Another object of the present invention is to provide a vessel for administration of anesthesia by means of a flexible expandable tube that can be positioned correctly without interrupting gas flow and/or organ activity of a surgical patient.

It is also an object of the invention to provide a tube that can operate as an artificial flexible expandable vessel, such as a segment of a blood vessel to replace clogged arteries, or as a permanent catheter duct for providing fluids to or from the body.

It is also an object of the present invention to improve over the disadvantages of the existing prior art tubes.

The basic concept of the present invention is to equip an inter vivos vessel, such as an endotracheal tube, artificial blood vessel or other tube, with a positioning mechanism that is activated from a proximal end of the vessel and allows exact positioning and reversible anchoring within a body cavity, such as the trachea. The expandable tubes disclosed herein can also be utilized as esophageal dilators, laparoscopic tubes, etc.

In the endotracheal tube embodiment, exact positioning and anchoring provide the conditions to provide anesthetic gases at the target, namely to the bronchial tubes, and the lungs.

In the present invention, the endotracheal tube can be anchored in the internal diameter of a body cavity, such as the trachea. The tube is expanded in size by means of an axially and longitudinally extendable elements inserted within the opposite free ends of a cul-de-sac formed by an H-shaped member. The extendable member includes free ends that run substantially the longitudinally length of the intro vivos tube. The two free edges of the extendable (flexible) cylindrical body elements engage corresponding free ends (cavities) of the H-shaped member, which is curved to complete the circumference of the flexible expandable endotracheal tube. The “H” segment also provides for the integrity of the tube and is constructed of a more rigid plastic than the rest of the tube itself. The remainder of the endotracheal tube utilizes the same or similar semi-rigid materials used in conventional inter vivos tubes. Polyvinyl tubes are presently used and continue to be used with varying degrees of hardness.

Moreover, upon extubation of the inter vivos tube of the present invention, retraction of the diameter of the tube is possible but not required. By axially shifting the segmented arches away from each other at the free ends of the tube within cul-de-sacs (cavities) of the “H” shaped element, the segmented arches are expanded so that the size of the endotracheal tube is increased and anchored during the administration of anesthesia. The segmented arches can be spread axially and longitudinally away from each other at the free ends thereof.

The free ends of the flexible cylindrical tube are axially and longitudinally displaced away from each other so that the internal diameter of the endotracheal tube is expanded to anchor the tube within a body cavity, such as the trachea.

It is important to note an expandable membrane 450 is sealed to the outer surface (403) of the “H” member element 401 and also completely surrounds the free ends or arms of the H-shaped member element. The portion of the membrane that surrounds the free arms of the H-shaped member allows the opposite free longitudinal ends of the endotracheal tube to remain inserted within the cul-de-sac formed by the free arms of the “H” element.

In another aspect of the invention, an expandable inter vivos tube comprising an H shaped member forming first and second cavities, a flexible longitudinal tube member having a first free end and an opposing second free end, the first free end slidably engaging the first cavity and the second free end slidably engaging the second cavity, a first expansion member contained in the first cavity between the rib member and the first free end and a second expansion member contained in the second cavity between the rib member and the second free end, the first expansion device and the second expansion device comprising a slideable member and an attached linking element, a sliding means engaging the slidable member in each of the first cavity and said second cavity, the sliding means operable between a first position of a slot in the H-shaped member and a second position of the slot.

In another aspect of the invention, the entire endotracheal tube can, itself, be sealed by a condom-like membrane to maintain smoothness, integrity of the tube itself and also prevent leaks.

According to an embodiment of the invention, the free end of one side of the cylindrical body, or segmented arch, can be moved, and the opposite side may be firmly attached inside the H-shaped member. By means of the self-acting spreading of the endotracheal tube after insertion into the patient, the position of the endotracheal tube is maintained so that controlled anesthesia can be performed without gas regurgitation.

In another embodiment of the invention, an expandable inter vivos tube system is disclosed including an inter vivos tube comprising a first H shaped member forming first and second cavities, a flexible longitudinal tube member having a first free end and an opposing second free end, the first free end slidably engaging the first cavity and the second free end slidably engaging the second cavity, a first expansion member contained in the first cavity between the rib member and the first free end and a second expansion member contained in the second cavity between the rib member and the second free end, the first expansion device and the second expansion device comprising a fixed member and a slideable member slidably engaging the fixed member and a sliding means engaging the slidable member in each of said first cavity and said second cavity, the sliding means operable between a first position of a slot and a second position of said slot, wherein the slot is represented as a track in a rib of the H-shaped member.

In another embodiment of the invention, a distance between the fixed member and the slidable member increases as the sliding means traverses between the first position and the second position.

In another embodiment of the invention, a hollow screw-type cap may be screwed onto the “H” shaped element to retain the sliding means in a desired position.

In another aspect of the invention, the elasticity of the tube member may provide sufficient pressure (i.e. force) to enable the free ends of the endotracheal tube to retract into the cul-de-sac(s) of the H-shaped element.

The inter vivos tube system of the present invention, advantageously, expands substantially uniformly along its entire axial length by increasing a distance between free ends of a slit in the inter vivos tube.

### BRIEF DESCRIPTION OF THE DRAWINGS

The advantages, nature, and various additional features of the invention will appear more fully upon consideration of the illustrative embodiments to be described in detail in connection with accompanying drawings wherein like reference numerals are used to identify like element throughout the drawings:

FIG. 1 illustrates a prospective view of conventional endotracheal tube with an expanded distal cuff which compresses distally against the tracheal wail.

FIG. 2 illustrates a prospective view of a conventional tracheostomy tube inflated distally in the same manner, as in FIG. 1.

FIG. 3 illustrates a prospective view of a conventional endotracheal tube inserted through the vocal cords and expanded within the trachea.

FIGS. 4A and 4B illustrates cross-sectional views of a first and second aspect of inter vivos tubes in accordance with the principles of the invention.

FIGS. 5A and 5B illustrate two prospective views of the inter vivos tube in accordance with a first embodiment of the invention.

FIGS. 6A and 6B illustrate a first exemplary expansion means in accordance with the principles of the invention.

FIGS. 7A and 7B illustrate a second exemplary expansion means in accordance with the principles of the invention.

FIGS. 8A and 8B illustrate a third exemplary expansion means in accordance with the principles of the invention.

FIG. 9 illustrates a prospective view of an inter vivos tube in accordance with another aspect of the invention.

FIG. 10 illustrates a prospective view of an inter vivos tube in accordance with the principles of the invention.

It is noted that the drawings of the invention are not to scale. The drawings are intended to depict only typical aspects of the invention, and therefore should not be considered as limiting the scope of the invention. In the drawings, like numbering represents like-elements among the drawings.

It is to be understood that the figures and descriptions of the present invention described herein have been simplified to illustrate the elements that are relevant for a clear understanding of the present invention, while eliminating, for purposes of clarity many other elements. However, because these omitted elements are well-known in the art, and because they do not facilitate a better understanding of the present invention, a discussion of such elements is not provided herein. The disclosure herein is also directed to variations and modifications known to those skilled in the art.

### DETAILED DESCRIPTION

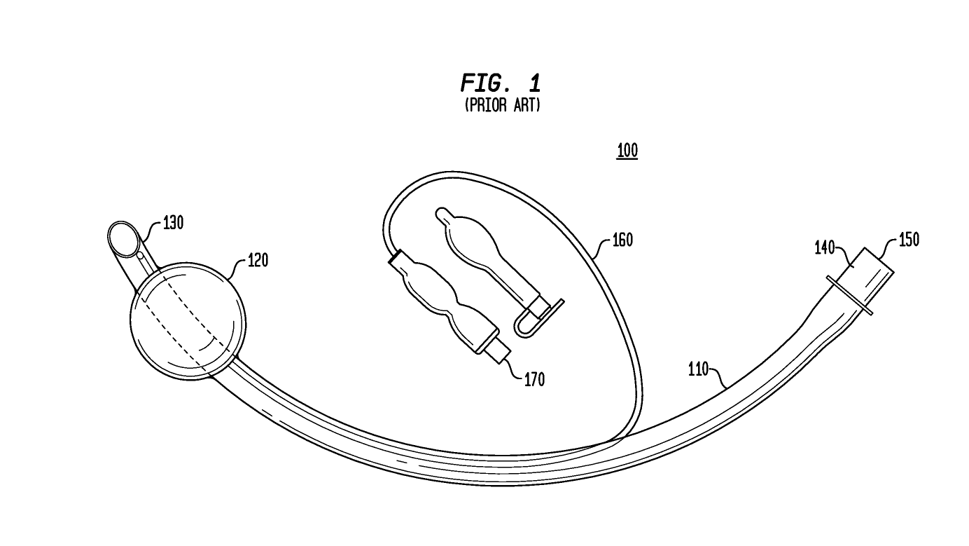

FIG. 1 illustrates a conventional endotracheal tube (i.e., inter vivos tube) 100 represented as an elongated tube 110 having a bulb member 120 positioned on a distal end 130 and a connection member 140 on a proximate end 150. The connection member 140 on proximate end 150 provides a means for allowing gases to flow through inter vivos tube 100 to distal end 130. Bulb member 120, which is shown in an expanded position, seals a passageway (not shown) into which inter vivos tube 100 is inserted to prevent gases exiting the distal end 130 from escaping along the inter vivos tube 100.

FIG. 1 further illustrates a smaller tube 160 running along an inner edge of inter vivos tube 100. Tube 160 may be used to provide a fluid, e.g., air, to bulb member 120 so as to expand bulb member 120 to the illustrated inflated position. Tube 160 may be connected to an air or liquid supply (not shown) by connection member 170.

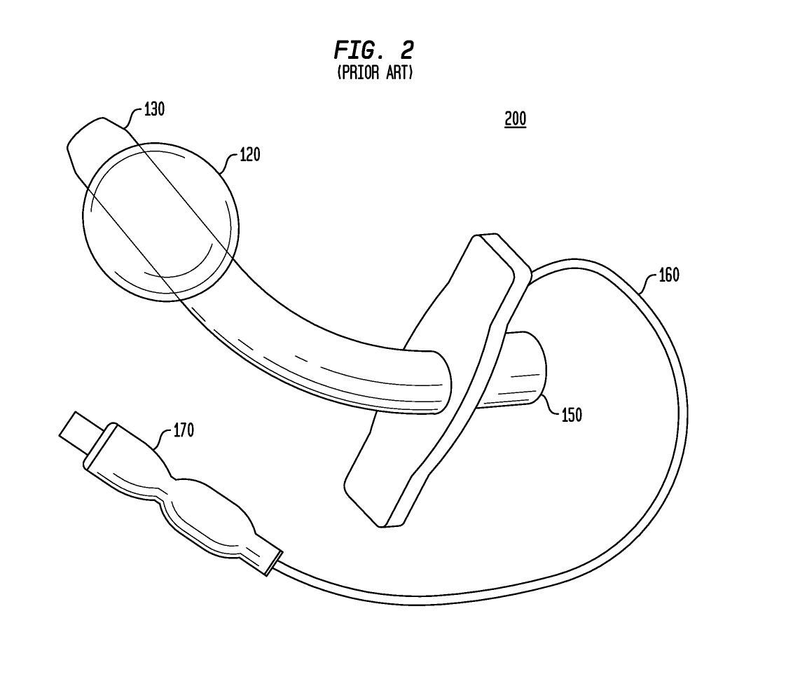

FIG. 2 illustrates a conventional tracheostomy tube (i.e., inter vivos tube) 200 used in providing air to a patient undergoing a tracheostomy process. Inter vivos tube 200 operates in a manner similar to that of the inter vivos tube 100 shown in FIG. 1, wherein a bulb member 120, positioned at a distal end 130, is expanded to prevent a fluid (e.g., air or liquid) injected into the proximate end 150 from escaping along the inter vivos tube 200. A fluid, such as air or liquid, enters through connection member 170 to expand bulb member 120, as previously discussed.

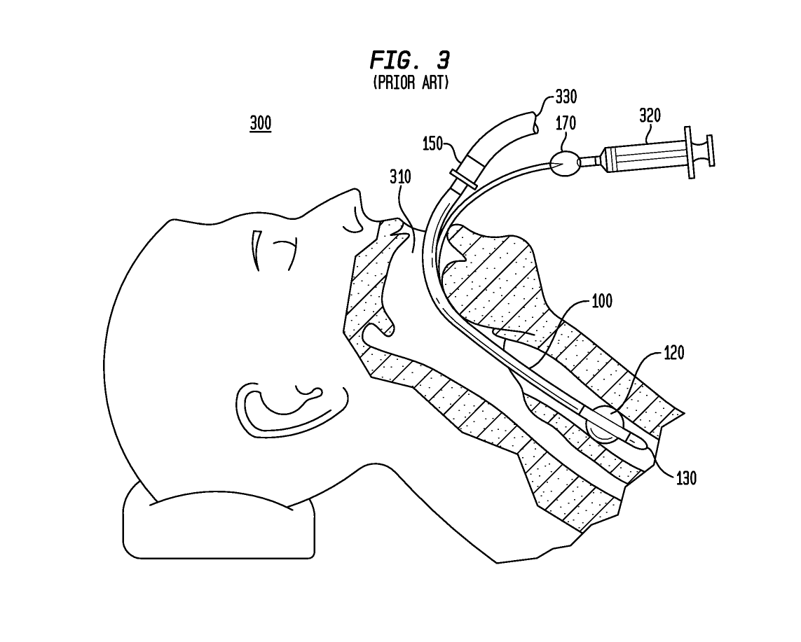

FIG. 3 illustrates a cross-sectional view 300 of the insertion and positioning of a conventional endotracheal tube 100 through a patient's vocal cords. As shown, bulb member 120 is an expanded mode to seal the patient's air passage 310. Also shown is syringe 320 that is connected to connection member 170 that represents a means for providing fluid to bulb member 120 so as to expand bulb member 120 to seal air passage 310. Also shown is tube 330 that is connected to connection member 170 to allow a fluid (e.g., gas, air, liquid) to pass from proximate end 150 of the inserted endotracheal tube 100 to distal end 130 of the inserted endotracheal tube 100.

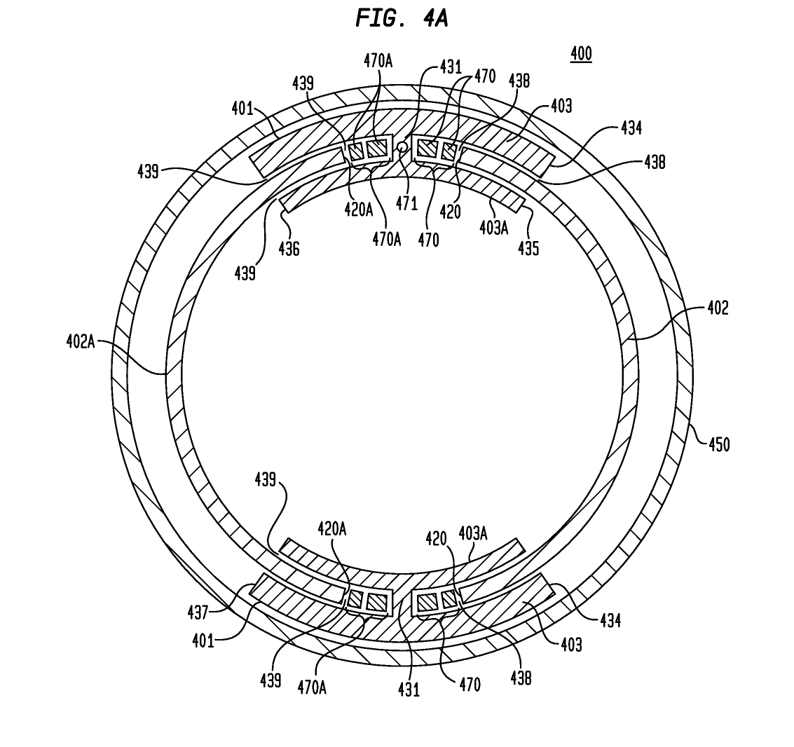

FIG. 4A illustrates a cross-sectional view of an exemplary inter vivos tube 400 in accordance with the principles of the invention. As shown, inter vivos tube 400 includes two (2) H-shaped connector member 401 opposite to each other and extending substantially longitudinally along an edge of inter vivos tube 400. Each of the H-shaped connector member 401 comprises an arched outer element 403 (outer circumference element) and an arched inner element 403A (inner circumference element) arranged circumferentially opposite each other at equal angles to each other along a circumference of the flexible expandable inter vivos tube 400. The H-shaped connector member 401 includes rib 431, which represents the cross-bar of the “H” in the H-shaped connection member 401 joining at a substantial midpoint of the arched elements 403 and 403A. The H-shape member 401 outer element 403 and inner element 403A, taken with rib 431, also form cul-de-sac receptacle cavities 438 and 439, respectively. The cul-de-sac cavities 438 and 439 have an opening that is sized to receive, in tongue-in-groove-like fashion, free end (i.e., tongue) portions 420 and 420A of arched tube segments 402 and 402A, respectively. The H-shaped connector member 401 has respective free ends 434, 435 that define cavity 438 and free ends 436 and 437 that define cavity 439. Outer curved or ached element 403 is longer than inner curved arched element 403A to accommodate an increase in circumference.

Rib 431 connects the arched elements 403, 403A of each H-shaped connecter member 401 and provides rigidity and structural integrity for the inter vivos tube 400. The rigidity of rib 431 has sufficient flexibility to enable the inter vivos tube 400 to be inserted into the trachea of the patient and to conform to the patient's airway, while retaining sufficient rigidity to permit a medical worker to position and to insert the tube 400 against anatomical resistance of the patient's throat and airway structures. Rib 431 may also include longitudinal conduit 471 for accepting a fiber optic cable for view-scope enablement.

The H-shaped connector member 401 may be made of a material such as polyvinyl chloride plastic, to provide sufficient rigidity and flexibility.

Tongues 420, 420A of arched tube elements 402, 402A, respectively, are normally in a retracted position within corresponding cavities 438, 439, providing inter vivos tube 400 with a minimum diameter.

Although not shown, it would be appreciated that the diameter of inter vivos tube 400, along an axis substantially perpendicular to the arched tube elements 402,402A increases when tongues 420, 420A are forced circumferentially apart. Hence, the cross sectional profile of the inter vivos tube 400 in accordance with the principles of the invention is one of substantially circular in an unexpanded mode and of an elliptical in an expanded mode. The configuration of the vocal cords themselves is not circular; assuming a somewhat triangular configuration.

The increased diameter of the inter vivos tube 400, caused by the displacement of the tongue elements 420, 420A of corresponding arched segments 402, 402A causes the passageway (FIG. 3, 310) into which the inter vivos tube 400 is inserted to become blocked, such that air may only enter or exit the passageway through the internal diameter formed by the inter vivos tube 400.

In addition, the cavities 438, 439 are sized to prevent tongues 420, 420A from expanding to a distance that would cause tongues 420, 420A to exit cavities 438, 439.

Also, shown is an, optional, expandable membrane (e.g., a condom) 450 that surrounds inter vivos tube 400. Optional membrane 450 may be composed of a material that provides for a smooth surface of the inter vivos tube 400. The optional membrane 450 may be composed of a material such as PVC (polyvinyl chloride) that allows for a smooth entry and exit of the inter vivos tube 400 into and out of a passage way (e.g., FIG. 3, 310).

Also illustrated are expandable members 470, 47A incorporated into cavities 438, 439, respectively. Expandable members 470,470A as described below, provide means for expanding inter vivos tube 400 by pushing tongues 420, 420A from cavities 438, 439 respectively.

Although the invention has been described with regard to incorporating two (2) H-shaped members 401 into the inter vivos tube that are substantially opposite to each other, it would be recognized that the positioning of the two H-shaped members 401 may be oriented at an angle different than substantially opposite without altering the scope of the invention.

FIG. 4B illustrates a second aspect of an inter vivos tube 480, which is similar to the inter vivos tube 400 shown in FIG. 4A. In this second aspect of the invention, a single H-shaped connection member 401 is incorporated into the inter vivos tube 480. As the elements of the single H-shaped member 401 shown in inter vivos tube 480 is the same as the H-shaped connector member 401 described with regard to FIG. 4A, a detailed description of H-shaped member 401 shown in FIG. 4B need not be repeated again herein.

In this exemplary second aspect, and as previously described, a diameter of the inter vivos tube 480 increases in a direction substantially perpendicular to arched segments 402, 402A as tongues 420, 420A are displaced from cavities 438, 439 by expandable members 470, 47A, respectively.

Although, FIGS. 4A and 4B illustrate H-shaped members having arched outer element 403 (outer circumference element) and an arched inner element 403A (inner circumference element) that are substantially parallel, it would be recognized that inner circumference element 403A and outer circumference element 303 may have radii that create a cul-de-sac cavities 438, 439 having a non-uniform size. That is, a distance between outer element 403 and inner element 403A at the opening of cavity 438 (439) is less than a distance between outer element 403 and inner element 403A at the rib 431.

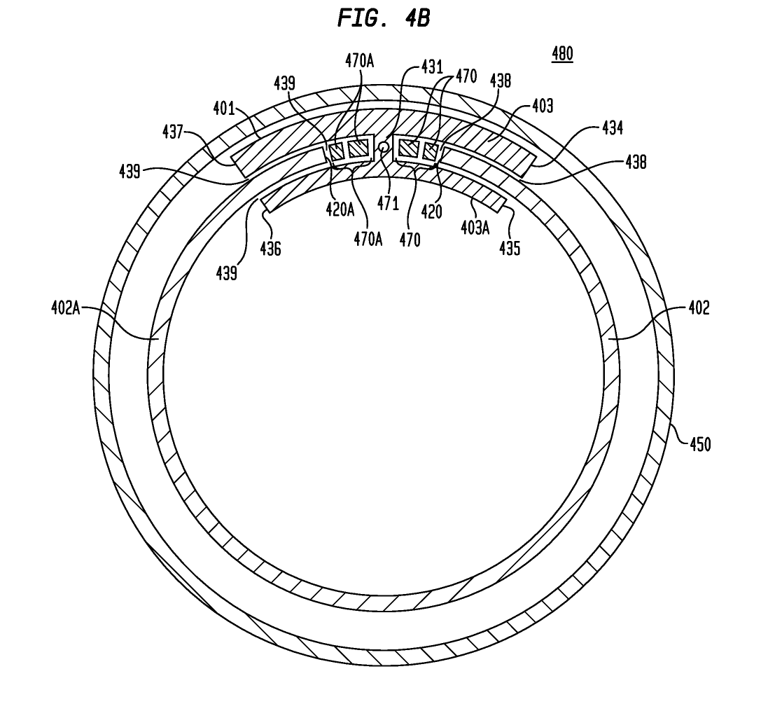

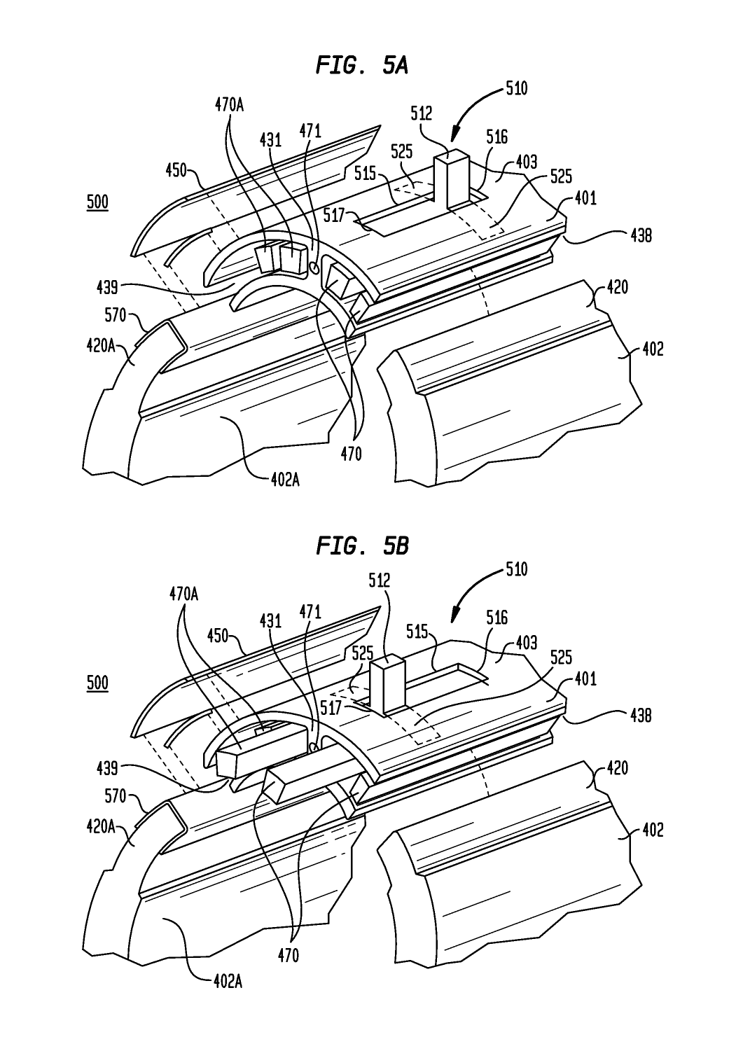

FIG. 5A illustrates a prospective cross-sectional view of a contracted inter vivos tube 400 (480) shown in FIG. 4A (FIG. 4B) wherein tongues 420, 420A are illustrated displaced from cavities 438, 439 in order to provide visual clarity to the subject matter claimed. Generally, in this contracted view tongues 420, 420A are contained within cavities 438, 439, respectively and are merely shown exited from cavities 438, 439 to show the operation of sliding means 470, 470A. In this exemplary embodiment shown, tongues 420, 420A may be tapered to allow easy entry or exit of tongues 420, 420A into cavities 438, 439, respectively.

Although tongues 420,420A are shown as being external to cavities 438, 439, respectively, it would be recognized that this positioning is merely to illustrate the invention claimed. Generally, upper arc member (outer circumference member) 403 and lower arc member (inner circumference member) 403A are sized to prevent tongues 420, 420A from exiting cavity 438, 439, respectively.

Also illustrated is slot 515 in outer arc member 403 extending from a first position 516 to a second position 517. Within slot 515 is sliding means 510, which includes a tab, nib or flap 512 attached to horizontal extensions 525, which are positioned along an inner surface of outer arc member 403. Second position 517 provides a limitation to the extent of travel of tab or nib 512. Horizontal extensions 525 are substantially perpendicular to the direction of travel of tab, nib or flap 512.

Sliding means 510 slides between first position 516 and second position 517 of slot 510 created within outer arc member 430. In this illustrated example, tab, lever, nib or flap 512 is shown resting against first position 516, which places tongues 402, 402A in a closed position; a minimum diameter inter vivos tube 400 (480).

Although tab, nib or flap 512 is shown extending from outer upper arc member 403 it would be recognized that the tab, nib, lever or flap 512 may also be an indentation in which an object (e.g., a finger, needle point, etc.) may be inserted into in order to move the indentation 512 from the first position 516 to the second position 517.

Sliding means 510 may slide along rib 431 on a track within rib 431, using, for example, a zip-lock mechanism. That is, rib 431 may have a track molded therein, either along its top surface or on either (or both) side surfaces. Sliding means 510 may then engage the track in rib 431 to slide from the first position 516 to the second position 517. In another aspect of the invention, rib 431, in an area corresponding to slot 515 may include an indentation slides along an upper surface of the indentation within rib 431.

In accordance with the principles of the invention, tongues 420, 420A within cavities 438, 439, respectively, extend as expansion means 470A, 470A is extended as sliding means 510 traverses from the first position 516 to the second position 571 of slot 515.

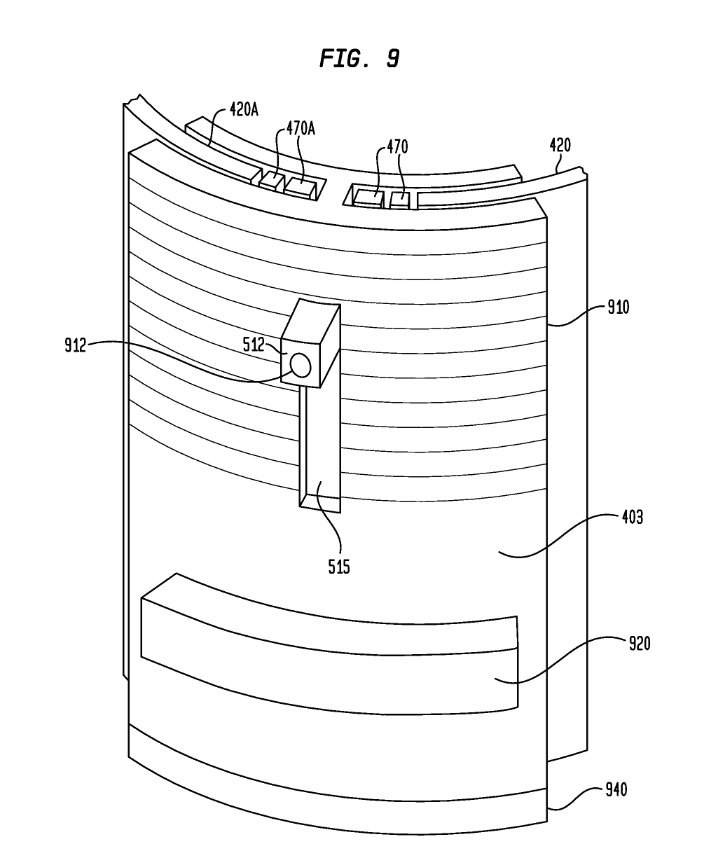

In another aspect of the invention, a fixed flap or lever or handle (see FIG. 9) may be positioned on external surface 403 distal to the second position 517. The fixed flap or level may be used to provide a position that may be held as lever 512 is moved from the first position 516 to the second position 517.

Also illustrated are expansion means 470 contained within cavity 438 and expansion means 470A contained within cavity 439. Expansion means 470,470A are attached to corresponding wings 525 of sliding means 510, such that as sliding means 510 (i.e., lever 512) traverses between the first position 516 to a position no greater than the second position 517, expansion means 470, 470A expand to cause corresponding tongues 420, 420A to be displaced (or extend) from cavities 438, 439, respectively. When flap or lever 512 reaches the second position 517 within slot 515 the extension means 470, 470A extend the edges to the tongues 420, 420A in cavities 438, 439, respectively, so that a maximum displacement of tongues 420, 420A is achieved.

FIG. 5B illustrates a prospective cross-sectional view of an inter vivos tube 400 (480) wherein the tab 512 is shown adjacent to the second position 517 of slot 515. In this illustrated example, the inter vivos 400 (480) is shown in an open position; a maximum diameter. Also illustrated in this open position, the expansion means 470, 470A are separated by a distance that is greater than the distance shown in the closed position shown in FIG. 5A. As is shown, as tab 512 slides within slot 515, a sliding portion of expansion means 470, 470A slides relative to a fixed portion of expansion means 470, 470A so as to expand the distance between the sliding member (portion) and the fixed member (portion).

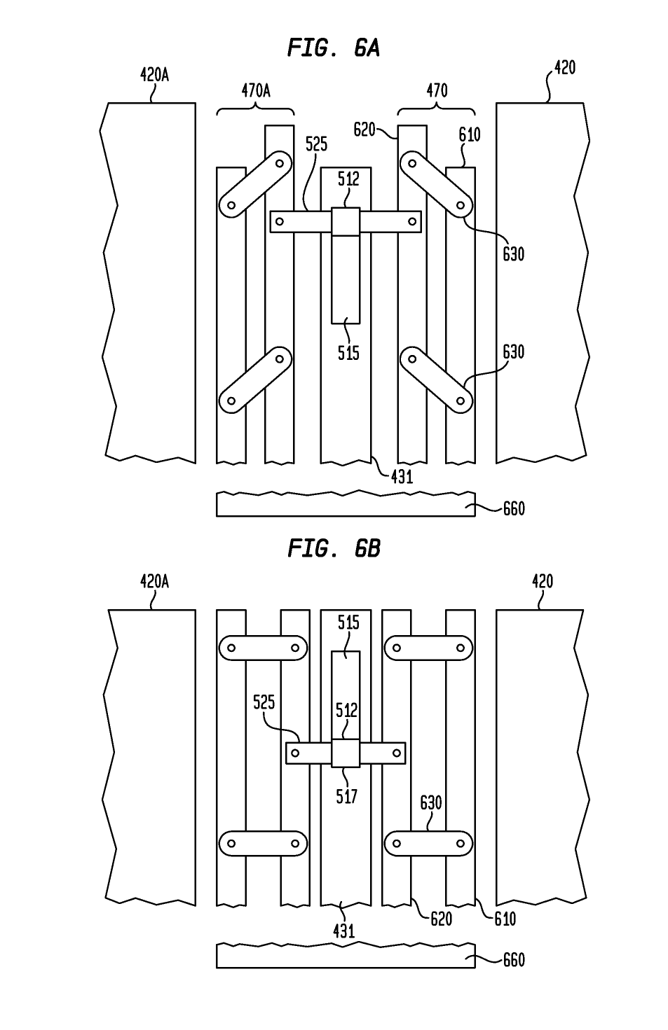

FIGS. 6A and 6B illustrate a first exemplary expansion means or device in accordance with the principles of the invention. FIG. 6A illustrates an exemplary expansion means 470, 470A incorporated into corresponding cavities 438, 439 separated by rib 431. In this case, expansion means 470, for example, includes a first element 620, adjacent to rib 431, and a second element 610, adjacent to tongue 420. First element 620 and second element 610 are joined by at least one link or hinge element 630; preferably, one link member 630 at a substantially proximal end of H-member 401 and one link member 630 at a substantially distal end of H-member 401. Expansion means 470A has a similar construction and need not be discussed in detail.

In another aspect of the invention, the fixed element of expansion means 470, 470A may be adjacent to, and in contact with, rib 431 and the sliding element of expansion means 470, 470A may be in contact with corresponding tongues 402, 402A. Also, it would be recognized that while a space is shown between rib 431 and expansion means 470, 470A, this spacing is presented only to provide clarity to the subject matter claimed as the invention.

Also shown is wing element 525 attached to first element 620 and tab or lever 512 in a first position in slot 515. In this case, contracted position, first element 620 and second element 610 are at a minimum distance from each other. Hence, corresponding tongues 420, 420A are in a first position and the inter vivos tube 400 is of a minimum diameter.

Also shown is a bottom edge 660 at a distal end of H-shaped member 401. Bottom edge 660 closes the distal end of H-shaped member 410 to inhibit expansion member 470, 470A from extending past the distal end of H-shaped member 401. Although bottom edge 660 is shown as a continuous element, it would be appreciated that bottom edge 660 may represent plug-type elements that are inserted within a distal end of cavities 438, 439 respectively.

FIG. 6B illustrates another aspect of the exemplary means 470, 470A shown in FIG. 6A. In this Illustrated aspect, lever 512 is positioned at the second position 517 of slot 515. In this case, first element 620, attached to lever 512 through wing element 525, correspondingly slides along rib 431, such that link or hinge element 630 traverses to a substantially horizontal position (i.e. perpendicular to rib 431). As link 630 is extended to a substantially horizontal position, second element 610 is extended outward to cause tongue 420, 420A to be displaced from cavity 438, 439, respectively. In this case, the inter vivos tube 400 (480) is of a maximum diameter.

In accordance with the principles of the invention, as sliding element 620 slides within a corresponding cavity (438, 439), the sliding element 620 interacts with a corresponding tongues 420,420A to cause the diameter of the inter vivos tube 400 (480) to increase. It would be recognized that the diameter of the inter vivos tube 400 (480) may be extended to any diameter between a minimum diameter (FIG. 6A) and a maximum diameter (FIG. 6B), depending upon the position of tab 512 within slot 515.

It would be recognized that the operation of expansion means 470A is similar to that described for expansion means 470 and, hence, a detailed discussion of the operation of expansion means 470A need not be repeated.

In accordance with the principles of the invention, as the distance between the fixed member 610 and the sliding member 620 increases, the corresponding tongues 420 (420A) slide outward and expand the diameter of the inter vivos tube 400 (480). Similarly, the distance between the fixed member 610 and the sliding member 620 may be decreased as a force applied to sliding member 620 is decreased and a force applied by tongue 420 (420A) causes the distance between the fixed member 610 and the sliding member 620 to decrease. The force applied by the tongues 420 (420A) may be determined based on an elasticity of the material of the inter vivos tube 400 (480).

Although FIGS. 6A and 6B illustrate the sliding member 620 being positioned closest to rib member 431, it would be appreciated that the sliding member 620 may be positioned closest to the tongues 420 (420A) and the fixed member 610 may be positioned closest to the rib member 431 without altering the scope of the invention.

In addition, it would be appreciated that a distal end of the H-shaped member 401 may be closed or sealed (See FIG. 9) to prevent any problems in case a pivot attachment 630 should become dislodged and/or to prevent the distal displacement of expansion member 470, 470A to limit the extension of tongues 420, 420A.

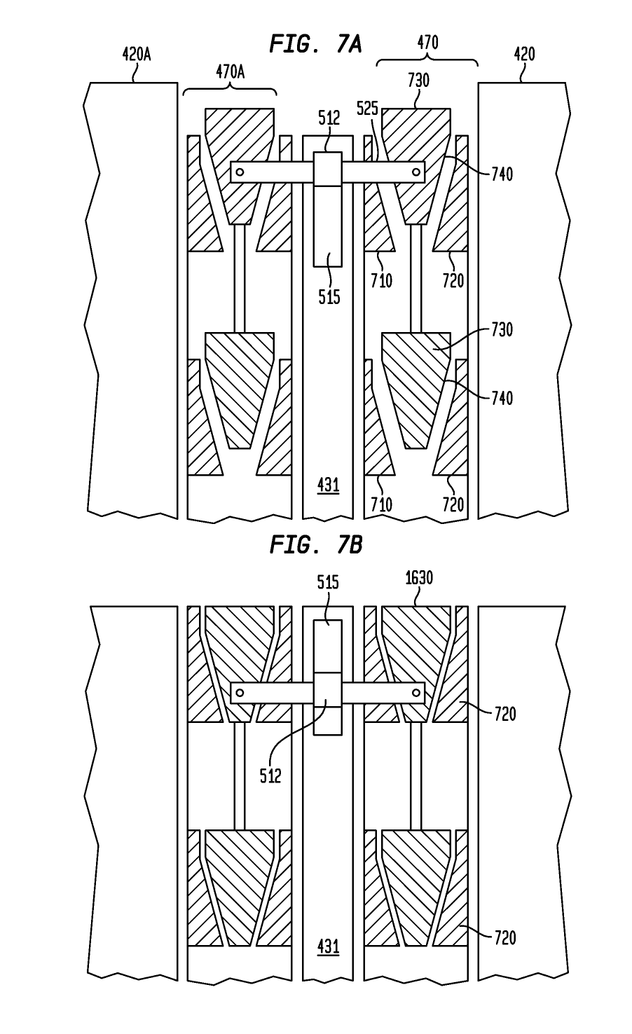

FIGS. 7A and 7B illustrate a second exemplary expansion device in accordance with the principles of the invention. In this illustrated example, the sliding mechanism 470 is comprised of a wedge shaped fixed member 710 that may be positioned on at least one side that is closest to rib member 431 or tongue 420 (420A). In the illustrated embodiment, one wedge shaped fixed member 710 is positioned closest to the rib member 431 and a second wedge shaped member 720 is positioned closest to the tongue 420 (420A). Also illustrated is a plunger shaped sliding member 730 that contains a wedge shape 740 that is opposite to that of the fixed member 710. The opposing wedge shapes 740 of sliding member 730 engage the wedged shaped fixed member 710, 720 to expand the inter vivos tube 400 (480). FIG. 7A illustrates an example in which the wedge shaped elements 740 of the sliding member 730 are not engaged with the fixed wedge shape members 710, 720. In this case, tab 512 in slot 515 is in a first position, as described with regard to FIG. 6A. FIG. 7B illustrates an example in which the wedge shaped elements 740 of the sliding member 730 engage the fixed wedge shape members 710, 720 to extend the tongues 420 (420A) away from rib member 431. In this engaged position, the diameter of the inter vivos tube 400 (480) is increased, similar to that shown in FIG. 6B.

It would be recognized that the wedge shaped elements 710, 720 may be formed directly onto at least one of the tongues 420 (420A) and rib member 431. In this manner, the expansion member 470 (470A) may be an integral element of illustrated inter vivos tube 400 (480). In another aspect of the invention, the wedge shaped elements 710, 720 and the sliding element 730 may be separately formed and then fitted within corresponding cavities 438, 439. The wedge shaped elements 710, 720 may be held in place with an adhesive applied to the fixed element.

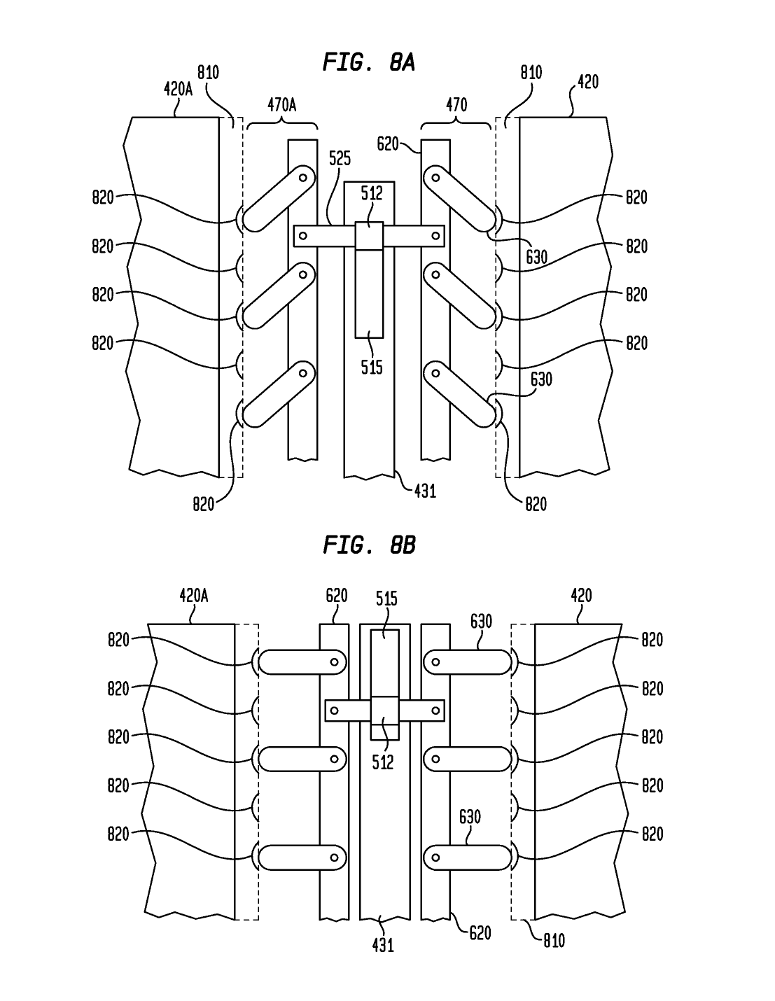

FIGS. 8A and 8B illustrate a third exemplary expansion device in accordance with the principles of the invention. In this illustrated example, which is similar to that shown in FIGS. 6A and 6B, expansion device 470 (470A) includes a sliding element 620 and at least one link element 630 pivotally attached, and extending from, the sliding element 620. The at least one link element 630 engages a medial edge of corresponding tongue 420 (420A) to extend corresponding tongue 420 (420A) to increase the diameter of inter vivos tube 400 (480) as sliding means 510 traverses the distance of slot 515 from a first position 516 to a second position 517. As would be appreciated, a free end of each of the at least one link elements 630 may be tapered in order to engage the medial edge of corresponding tongue 420 (420A).

The medial edges of tongues 420, 420A, which are positioned within cavities 438, 439, respectively, may be roughened or layered with a thin hardened strip 810 attached to the medial edges. Strip 810 may have a roughened surface or include sockets or indentation 840. The roughened surface or indentations 820 (directly on medial edges of tongues 420, 420A or on strips 810) may be used by link elements 630 as engagement points to secure link element 630 to the medial edge of corresponding tongue 420 (420A). Strip 810 may be of a material comparable in hardness to the hardness of the link elements 630 and may be glued or fused onto the free ends of tongues 420, 420A, respectively. In another aspect, the FIG. 8B, similar to FIG. 6B, illustrates the case wherein the lever 512 is moved to second position 517 in slot 515 to expand tongues 420 (420A) to a maximum extension. In this case, link elements 630 are substantially vertical (i.e., perpendicular) to rib 431, as previously discussed.

As would be recognized, the expansion device 470 (470A) shown in FIG. 8A may be formed external to the cavity 438 (439) and inserted into corresponding cavity 438 (439).

Although FIGS. 8A and 8B illustrate links 630 as being slidable engaging with the medial edges of tongue 420, 420A, whether with or without strips 810, it would be appreciated that the links 630 may be pivotally attached to tongues 420, 420A (i.e., medial edges of elements 402, 402A, respectively). Such pivot attachment may be similar to that shown in FIGS. 6A and 6B. In this case, strips 810 may represent the fixed portion of the expansion means 470, 470A shown in FIG. 6A.

In addition, strips 810 may be constructed in a manner to include a “U-shaped” channel, such that strip 810 may be slide onto tongues 420, 420A (see for example, element 570, FIG. 5). Strip 810 may be held in place by friction and/or an adhesive and/or fused to the corresponding tongue 420, 420A.

In accordance with the principles of the invention, expansion means 470, 470A including slidable element 620, links 630, and strips 810, pivotally attached to links 630, may be formed external to the cavity 438, 439 and then slide into place by sliding strip 810 over tongues 420, 420A.

FIG. 9 illustrates a perspective view of an inter vivos tube 400 in accordance with another embodiment of the invention. In this exemplary perspective view, a screw tread 910 extends along a portion of the outer surface 403. Also shown is an expansion device 470, 470A contained within corresponding ones of cul-de-sac cavities 438, 439 in H-shaped member 401. The expansion device 470, 470A, as discussed, provides means for expanding a diameter of flexible tube 400 (480) by sliding the tongues 420, 420A of the flexible tube 400 (480) away from the rib member 431 of H-shaped member 401. The expansion of the flexible tube 400 by displacing the tongues 420, 420A from the cavities 438, 439 has been previously discussed.

Screw thread 910 allows a hollow shaped adaptor (not shown) to be screwed onto H-shaped member in order to retain tab 512 in a desired position.

In accordance with the principles of the invention, after the tab or lever 512 achieves a desired position and the inter vivos tube 400 (480) is expanded to a desired diameter, air may be introduced to the patient through the expanded inter vivos tube.

Also shown is fixed arm 920 that is positioned distal to slot 510. Fixed arm 920 may be used to hold inter vivos tube 400 (480) as tab (or indentation) 512 is moved from the first position 516 to the second position 517. Also shown at a distal end of H-shaped element 401 is bottom element 940 that is used to seal the end of H-shaped member 401. The sealing of the distal end of H-shape element 401 is advantageous in that it insures that sliding means 470 does not extend past the distal end of the H-shaped member 401.

Also shown is recess 912 in tab 512. Recess 912 includes an internal screw thread (not shown) that extends through tab 512. The screw thread, within tab 512, positions a screw (not shown) within recess 912 to engage rib 431. Engaged screw retains tab 512 in place. The screw may be, for example, a set screw, that may be turned using a screw driver or an Allen key. Alternatively, the screw may have a significantly large head (e.g., a thumb screw) such that the screw may be turned between a thumb and forefinger.

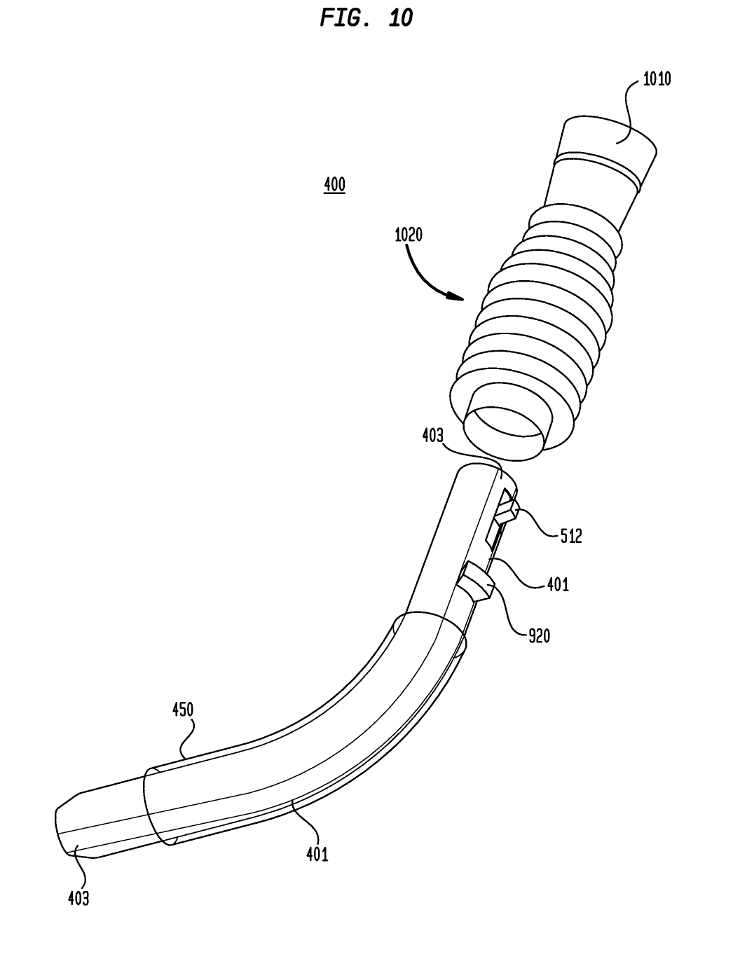

FIG. 10 illustrates a perspective view of an exemplary inter vivos tube 400 (480) in accordance with the principles of the invention. In this exemplary inter vivos tube 400 (480), H-shaped member 401 extends substantially along the longitude axis of the inter vivos tube 400 (480). Sliding means 512 is shown within slot 515 and fixed arm 920 is also shown. Membrane 450 surrounding inter vivos tube 400 (480) is shown covering the insertable portion of inter vivos tube 400 (480).

Also shown is an air tube 1010 that engages the exemplary inter vivos tube 400 (480) disclosed herein. Air tube 1010 includes an accordion type attachment 1020 that engages a proximate end of inter vivos tube 400 (480). Accordion type attachment 1020 may extend over sliding means 512 (i.e., down to fixed arm 920) in order to provide an air tight seal.

In one aspect of the invention, after the sliding means 512 is positioned, it may be taped in place to prevent further movement. In another aspect of the invention a cap (not shown) may be used to retain sliding means 512 in place after the sliding means 512 has been positioned to expand inter vivos tube 400 (480) to a desired width. The cap (not shown) may be slide over the proximate end of the inter vivos tube 400 (480) and held in place by a tape or Velcro-type attachment means. Alternatively, when the H-shaped member includes a screw thread, as shown in FIG. 9, the cap may include an internal thread that is screwed into position and may be held in place by the friction of the screw thread. The accordion type attachment 1020 may then be placed over the cap, down to fixed arm 920, in order to provide an air tight connection to avoid the loss of air directed through inter vivos tube 400 (480). In another aspect of the invention, a gel type material (e.g., a jelly, VASOLINE, etc.) may be applied to slot 515, after tab 512 is slide into position, to provide a further seal to avoid a loss of air through slot 515. Alternatively, the condom 450 may be drawn over the flange 920 to encompass the slot 515 and tab 512 to provide a seal to prevent a loss of air through slot 515.

While there has been shown, described, and pointed out fundamental and novel features of the present invention as applied to preferred embodiments thereof, it will be understood that various omissions and substitutions and changes in the apparatus described, in the form and details of the devices disclosed, and in their operation, may be made by those skilled in the art without departing from the spirit of the present invention.

The terms “a” or “an” as used herein are to describe elements and components of the invention. This is done merely for convenience and to give a general sense of the invention. The description herein should be read to include one or at least one and the singular also includes the plural unless indicated to the contrary.

The term “comprises”, “comprising”, “includes”, “including”, “as”, “having”, or any other variation thereof, are intended to cover non-exclusive inclusions. For example, a process, method, article or apparatus that comprises a list of elements is not necessarily limited to only those elements but may include other elements not expressly listed or inherent to such process, method, article, or apparatus. In addition, unless expressly stated to the contrary, the term “or” refers to an inclusive “or” and not to an exclusive “or”. For example, a condition A or B is satisfied by any one of the following: A is true (or present) and B is false (or not present); A is false (or not present) and B is true (or present); and both A and B are true (or present).

It is expressly intended that all combinations of those elements that perform substantially the same function in substantially the same way to achieve the same results are within the scope of the invention. Substitutions of elements from one described embodiment to another are also fully intended and contemplated.

What is claimed is:

1. An expandable inter vivos tube system comprising:
an inter vivos tube comprising:
a first H shaped member comprising:
an outer circumference member having a slot contained therein;
an inner circumference member; and
a rib element connecting said outer circumference member and said inner circumference member at substantially a midpoint of said outer circumference member and said inner circumference member, said rib member, an inner surface of said outer circumference member and a surface of said inner circumference member opposite said inner surface of said inner surface of said outer circumference member forming first and second cavities, respectively;
a flexible longitudinal tube member having a first slit along a longitudinal axis, said first slit creating a first free end and an opposing second free end, said first free end slidably engaging said first cavity and said second free end slidably engaging said second cavity;
a first expansion member contained in the first cavity between the rib and the first free end;
a second expansion member contained in the second cavity between the rib and the second free end, each of the first expansion device and the second expansion device comprising:
a slideable member interacting with a corresponding one of the first free end and said second free end; and
a sliding means engaging said slidable member in each of said first cavity and said second cavity, said sliding means operable between a first position of said slot and a second position of said slot.

2. The inter vivos tube of claim 1, wherein each of said first expansion member and said second expansion member further comprising:
at least one linking element attached to and extending from, said slidable member, each of said at least one linking element having a pivotable attachment at a first end of said linking element with said slidable member.

3. The inter vivos tube system of claim 1, further comprising:
an end piece closing a distal end of said first “H” shaped member.

4. The inter vivos tube system of claim 1, wherein said slidable member in said first cavity and said slidable member in said second cavity are adjacent to said rib.

5. The inter vivos tube system of claim 1, wherein said slidable member in said first cavity is adjacent to said first free end and said slidable member in said second cavity is adjacent to said second free end.

6. The inter vivos tube system of claim 1, further comprising:
a fixed member in each of said first cavity and said second cavity.

7. The inter vivos tube system of claim 6, wherein said fixed member and said slidable member are substantially opposite each other.

8. The inter vivos tube system of claim 1, wherein said sliding means comprises one of: a tab extending through said slot and an indentation accessible through said slot.

9. The inter vivos tube system of claim 6, wherein said fixed member is wedge shaped and attached to at least one of said rib and corresponding free end.

10. The inter vivos tube system of claim 9, wherein said sliding member is wedge shaped, said wedge shape being opposite to said wedge shape of said fixed member

11. The inter vivos tube system of claim 6, wherein said fixed member is pivotally attached to said linking element at a second end of said linking element.

12. The inter vivos tube system of claim 1, further comprising:
a thin strip attached to each of said first free end and said second free end.

13. The inter vivos tube system of claim 12, further comprising:
at least one indentation in said thin strip attached to each of said first free end and said second free end.

14. The inter vivos tube of claim 12, wherein said thin strip is pivotally attached to a corresponding one of the slidable member in the first cavity and the second cavity.

15. The inter vivos tube of claim 1, further comprising:
a screw tread on an outer surface of said outer circumference member.

16. An inter vivos tube comprising:
a flexible tube, split along a longitudinal axis, said split forming a first free end and a second free end;
an H-shaped member comprising:
an outer member having a slot extending therethrough;
an inner member; and
a rib member connecting, at a substantial mid-point, said outer member and said inner member, said outer member, said inner member and said rib member forming first and second cavities, respectively, wherein said first end and second free end slidably engage a corresponding one of said first cavity and second cavity;
an expansion device positioned in each of said first cavity and said second cavity between the rib member and corresponding first free end and second free end, said expansion device comprising:
a slidable member; and
at least one hinge element pivotally attached to said slidable member; and
a sliding mechanism slidably engaged to said rib through said slot and attached to the slidable member in each of said first cavity and said second cavity, said sliding mechanism traversing between a first position and a second position of said slot, wherein a distance between said rib member and said first free end and said rib member and said second free end increases as said sliding mechanism causes said slidable member to interact with said first free end and said second free end.

17. The inter vivos tube of claim 16, further comprising:
a membrane surrounding said inter vivos tube.

18. The inter vivos tube of claim 16 wherein said rib member further comprising one of:
a track in one of an upper surface and a side surface and a molded indentation in an upper surface.

19. The inter vivos tube of claim 16, further comprising:
a fixed member in each of said first cavity and said second cavity.

20. The inter vivos tube of claim 18,
wherein said fixed member and said slidable member have opposing wedge shapes.

21. The inter vivos tube of claim 18, wherein said fixed member is connected to said sliding member through said at least one hinge element.

22. The inter vivos tube of claim 16, further comprising:
a thin strip on each of said first free end and said second free end.

23. The inter vivos tube of claim 16 further comprising:
a screw thread on an outer surface of said outer member.

24. The inter vivos tube of claim 16, wherein said sliding means comprises one of: a tab extending through said slot and an indentation accessible through said slot.

25. The inter vivos tube of claim 16 further comprising:
a fixed arm distal from said second position of said slot.

26. The inter vivos tube of claim 16, further comprising:
an end piece closing a distal end of said “H” shaped member.

27. The inter vivos tube of claim 22, wherein said thin strip is pivotally attached to a corresponding one of said sliding mechanism in said first cavity and said second cavity.

28. The inter vivos tube of claim 27 wherein said thin strip is U-shaped, said U-shape engaging corresponding ones of said first free end and said second free end.
